(19) United States
(12) Patent Application Publication
(10) Pub. No.: US 2026/0151258 A1
(43) Pub. Date: Jun. 4, 2026

(54) FEMALE GENITALIA SHEATH APPARATUS

(71) Applicant: JOSE SANDOVAL, Caguas, PR (US)

(72) Inventor: JOSE SANDOVAL, Caguas, PR (US)

(21) Appl. No.: 19/388,870

(22) Filed: Nov. 13, 2025

Related U.S. Application Data
(63) Continuation-in-part of application No. 18/195,671, filed on May 10, 2023, now Pat. No. 12,491,107.
(60) Provisional application No. 63/340,508, filed on May 11, 2022.

(51) Int. Cl. A61F 6/06

(52) U.S. Cl. CPC A61F 6/065

(57) ABSTRACT

A female genitalia protective sheath apparatus is disclosed for providing a flexible, anatomically conforming, and hygienic barrier over external female genitalia. The apparatus includes a multilayer structure comprising a stimulus pad positioned between first and second protective layers, each supported by adhesive frames and removable covers to maintain sterility prior to use. The apparatus may further include an almond-shaped contoured frame, a semi-covered access region, a tear-away perimeter for controlled removal, and an optional magnetic coupling system for repeatable attachment. A handle facilitates positioning and removal while minimizing contact with the barrier surface. During use, the apparatus adheres securely to the skin, conforming to body movement while preventing fluid exchange and reducing the risk of pathogen transmission. The device provides an ergonomic, disposable, and female-oriented protective solution suitable for personal and clinical applications.

### BACKGROUND OF THE INVENTION

### Claim of Priority

This application is a Continuation-In-Part (CIP) of United States Non-Provisional patent application Ser. No. 18/195,671 , filed on May 10, 2023, which in turn claims the benefit of U.S. Provisional patent application Ser. No. 63/340,508 filed on May 11, 2022, both of which are incorporated by reference herein in their entireties.

### FIELD OF THE INVENTION

The present invention relates generally to prophylactic devices and systems for disease prevention and sexual health protection, and more particularly to a female genitalia protective sheath apparatus configured to provide barrier protection and optional structural variations for enhanced fit and functionality.

### DESCRIPTION OF THE RELATED ART

Barrier methods of disease prevention play an essential role in the broader field of public health and sexual wellness. Historically, such methods have focused primarily on male users, particularly through the use of condoms designed to cover the penis and prevent exchange of bodily fluids. While effective, this approach leaves a structural and functional gap in protection for female recipients of oral sex and related sexual activities. Despite ongoing advances in prophylactic materials and awareness campaigns, options specifically engineered to protect the external female genitalia remain limited, under-utilized, and often inadequate for consistent or comfortable use.

Commercially available female prophylactics, including female condoms and dental dams, generally operate by forming a flexible barrier over the vaginal opening or vulvar surface. However, most existing devices were not originally conceived for ergonomic adaptation to female anatomy. Dental dams, for example, are flat latex or polyurethane sheets that must be manually held in place during use. This design lacks structural stability, is easily displaced, and creates difficulties in maintaining contact coverage throughout movement. Female condoms, although designed for internal vaginal insertion, are not well suited for oral sexual activity because they are typically anchored internally rather than externally, and their cylindrical geometry fails to provide consistent external coverage of the labial and perineal areas.

Another recurring limitation of conventional female barrier devices is the conflict between reliable adhesion and user comfort. To remain in place, the device must establish a continuous seal along irregular skin contours that vary significantly between individuals. Achieving this seal without introducing discomfort, air pockets, or loss of sensation presents a persistent design challenge. Additionally, the adhesives used in existing products can lose effectiveness when exposed to moisture, perspiration, or the natural lubricants present during sexual activity. Some adhesives may cause irritation or allergic reactions, particularly when compounded with latex-based materials or certain bonding agents. These factors discourage consistent use, reducing the practical effectiveness of existing prophylactic devices.

Material selection further complicates this issue. Latex, though widely available and inexpensive, is associated with allergy risk and environmental degradation concerns. Alternative polymers such as polyurethane or polyisoprene provide improved biocompatibility and transparency but often exhibit lower elasticity and weaker adhesion properties. Moreover, thin films optimized for tactile sensitivity can tear or wrinkle under tension, leading to coverage gaps. A structurally stable yet comfortable barrier device must therefore achieve a delicate balance between flexibility, conformability, and mechanical strength.

From a geometric standpoint, most female barrier devices adopt a circular or oval shape intended to approximate the general outline of the vulvar region. In practice, however, these shapes often fail to maintain proper positioning once applied. The presence of motion, body curvature, and differing anatomical dimensions can cause the edges to peel, roll, or shift, compromising the seal. Some experimental configurations have attempted to introduce support rings or reinforced borders, but these modifications can increase rigidity and discomfort, particularly at contact points near sensitive tissue. The need for a frame geometry that provides reliable retention while maintaining gentle conformity remains unmet.

Accessibility during use presents another engineering challenge. For hygienic or functional reasons, a user or partner may need limited access to specific regions of the genitalia without removing the entire barrier. Existing devices offer no practical means of controlled access; removal or repositioning generally results in loss of adhesion and contamination of the adhesive surface. A device that allows selective entry or layered coverage, while maintaining continuous protection around the remaining perimeter, would enhance both safety and usability. Structural innovations that introduce semi-covered openings or overlapping film layers could allow such controlled interaction while preserving the overall barrier function.

Ease of removal and disposal also influences acceptance. Thin polymer films with smooth edges can adhere tightly, making removal awkward or even painful, particularly when combined with body moisture. Current products rarely include any dedicated tear-away or peel-assist feature. Users often resort to pulling or scraping along the skin, risking discomfort and contamination. An engineered edge configuration—such as an indented or perforated margin—could facilitate removal by allowing the user to deliberately initiate tearing along a predictable line without compromising barrier integrity during use.

Attachment and reusability have likewise emerged as topics of interest. While most sexual health products are intended for single use, prototype research has explored semi-reusable barrier frames that allow replacement of only the central protective film. Achieving detachable yet secure coupling between components presents technical challenges, particularly when flexibility and sterilization are required. Magnetic or metallic coupling mechanisms offer one possible approach: a light magnetic frame could enable the protective sheath to lock into place during use and be separated easily afterward without reliance on adhesive strength alone. However, integrating such mechanisms demands precise weight management and careful material selection to prevent interference with comfort or flexibility.

The manufacturing process for female barrier devices introduces further limitations. Many existing methods rely on flat film cutting and lamination, which are cost-effective but poorly suited for three-dimensional anatomical conformity. Layer-over-layer fabrication, where successive material layers are bonded to achieve differential thickness or tactile response, allows more complex structures but increases production cost and requires new tooling. Innovations in this area have demonstrated that layering materials with varying elastic moduli can improve both structural stability and user sensation, yet no standardized process currently addresses these objectives in a commercial female protective apparatus.

Beyond structural concerns, social and behavioral factors compound the adoption barrier. Products that require assistance for placement, visible external components, or complex instructions face low compliance rates. Discreet, self-applicable, and ergonomically adaptive designs are more likely to achieve consistent use. Therefore, there exists a strong public health interest in developing a prophylactic device that is both functionally reliable and unobtrusive in application. Design considerations must account not only for infection prevention but also for the psychological and sensory experience of the user.

Environmental and regulatory considerations further reinforce the need for innovation. Disposal of latex-based products contributes to non-biodegradable waste, prompting exploration of sustainable polymers and manufacturing processes. Additionally, regulatory agencies such as the U.S. Food and Drug Administration (FDA) may impose constraints on product weight, magnetic components, or chemical adhesives. Such constraints underscore the importance of adaptable design architectures that can be selectively modified to satisfy regulatory feedback without redesigning the entire apparatus.

Taken together, these engineering, ergonomic, and material challenges highlight a persistent gap in the field of female sexual health protection. Existing devices often sacrifice comfort for stability or vice versa, failing to deliver a consistently reliable barrier that aligns with the anatomical and functional realities of female use. The evolution of user expectations and regulatory scrutiny demands next-generation designs that merge medical safety, ergonomic performance, and sensory acceptability.

Accordingly, there remains a clear and continuing need for an improved female genitalia protective sheath apparatus that addresses these shortcomings by providing greater structural adaptability, optional access configurations, improved adhesion and removal mechanisms, and enhanced comfort without compromising the prophylactic function. Such a device would contribute significantly to advancing sexual health technology and expanding protective options available to women worldwide.

### SUMMARY OF THE INVENTION

The present invention is directed toward a female genitalia protective sheath apparatus configured to provide a flexible, adhesive, and anatomically conforming barrier for reducing the transmission of pathogens and bodily fluids during oral sexual activity. The apparatus comprises a multi-layer structure including a stimulus pad positioned between opposing protective layers, each secured by an adhesive frame and removable cover to preserve sterility prior to use. The apparatus may further include a handle laterally connected to facilitate application and removal. In additional embodiments, the invention incorporates structural variations designed to enhance fit, comfort, and usability, such as frames with almond or rounded geometries for improved retention, a lower access gap or semi-covered entrance between protective layers to permit controlled anatomical access, an indented or perforated tear-away edge for simplified removal, and an optional magnetic or metallic frame coupling mechanism that enables selective detachment and reattachment of the sheath. These configurations collectively provide a versatile and user-adaptive prophylactic device suitable for various anatomical profiles and intended applications.

The present invention offers significant advantages over existing prophylactic systems and female barrier devices by combining ergonomic adaptability with practical functionality. Traditional flat or circular barriers are prone to displacement, discomfort, and inconsistent coverage, whereas the improved frame geometries of the present invention conform more naturally to the body's contours and maintain stable positioning throughout use. The introduction of a controlled access gap addresses the long-standing usability challenge of maintaining protection while allowing limited tactile interaction, thereby improving user acceptance. The tear-away or indented border design enhances removability and hygiene by permitting predictable and clean separation from the skin without excessive force. Moreover, the optional magnet-to-metal locking mechanism provides an alternative to adhesive-based retention, offering reusability and mechanical precision in alignment while reducing the risk of irritation from prolonged adhesive exposure. Collectively, these improvements advance the state of the art by offering a structurally stable, comfortable, and anatomically sensitive barrier device that promotes safer and more consistent protection for female users.

In a first implementation of the present invention, a female genitalia protective sheath apparatus is provided for establishing a flexible and anatomically conforming barrier over external female genitalia to promote hygienic protection during sexual activity. The apparatus includes a stimulus pad positioned between a first protective layer and a second protective layer, each of the protective layers being superposed onto opposing sides of the stimulus pad to define a multilayer barrier structure. A frame assembly is coupled to a periphery of at least one of the protective layers and supports a removable cover that maintains the apparatus in a sterile condition prior to use. A handle extends laterally from the stimulus pad and at least one of the protective layers to facilitate manual placement and removal of the apparatus. The frame assembly defines an enhanced peripheral architecture that may include geometrically contoured portions shaped to improve anatomical conformity and retention, an access region formed between adjacent protective layers to permit controlled anatomical access, a tear-away margin configured to enable predictable user-initiated removal, and an optional magnetic or metallic coupling mechanism that allows selective attachment and reattachment to a mating frame or support. Collectively, these features provide a secure, comfortable, and disposable barrier system adapted for ergonomic fit and enhanced user control.

In another aspect, the frame assembly of the protective sheath apparatus may define a generally almond-shaped outline dimensioned to follow external contours of the female anatomy and improve positional stability and coverage during use.

In another aspect, the frame assembly may define a rounded-end configuration having a flatted lower region that can enhance comfort and anatomical fit when positioned between adjacent body surfaces.

In another aspect, the frame assembly may be formed from a semi-rigid or elastomeric polymer material that provides sufficient flexibility for application while maintaining elastic memory to return to its original shape after deformation.

In another aspect, the apparatus may include a lower access region disposed at a lower portion of the frame assembly, the access region defining an open gap between portions of the protective layers to permit controlled anatomical access.

In another aspect, the access region may be configured as a semi-covered entrance defined by overlapping edge portions of the first and second protective layers, allowing limited tactile interaction while maintaining peripheral sealing and hygienic protection.

In another aspect, the access region may incorporate an interior reinforcement border or bonded edge structure that can prevent tearing or delamination of the protective layers during movement or removal.

In another aspect, the apparatus may include a tear-away perimeter having a sequence of perforations or weakened score lines formed along a portion of the frame assembly to facilitate predictable user-initiated removal after use.

In another aspect, the tear-away perimeter may be indented or scored such that peeling or pulling along the margin initiates a controlled separation without compromising barrier integrity during wear.

In another aspect, the apparatus may include a detachable coupling mechanism integrated within the frame assembly, the coupling mechanism comprising magnetic and metallic elements that may enable selective attachment and reattachment to a mating frame, garment, or support structure.

In another aspect, the magnetic component may be formed of materials such as neodymium, samarium-cobalt, or ferrite encapsulated within a polymeric housing to ensure comfort and corrosion resistance.

In another aspect, the magnetic component may be arranged in a polarized orientation configured to guide automatic alignment and seating of the apparatus in a predefined operational position relative to a complementary frame or support.

In another aspect, the frame assembly may further include an adhesive layer positioned beneath the magnetic or metallic elements to enhance sealing contact with the user's skin surface and reduce potential displacement.

In another aspect, the multilayer assembly including the frame, protective layers, and stimulus pad may be fabricated through a lamination, bonding, or heat-sealing process that can provide a unified and fluid-resistant barrier structure.

In another aspect, the protective layers may be formed from transparent or translucent biocompatible polymer films that can allow visual observation of the covered region and ensure user comfort.

In another aspect, the stimulus pad may include a textured or patterned inner surface that can generate a tactile or sensory stimulus through the protective layers during contact.

In another aspect, the handle may be integrally formed with the frame assembly as a continuous molded extension that can facilitate grasping, placement, and removal of the apparatus without direct contact with the protective layers.

In another aspect, the frame assembly, protective layers, and removable cover may collectively define a disposable barrier unit that can be pre-sterilized and packaged for single-use application, ensuring hygienic handling and simplified disposal.

In another implementation of the present invention, a method is provided for applying a female genitalia protective sheath apparatus to establish a hygienic and conforming barrier over external female genitalia. The method includes providing a protective sheath apparatus comprising a stimulus pad positioned between a first protective layer and a second protective layer, the protective layers being coupled to a peripheral frame assembly and covered by a removable cover. The method further includes removing the removable cover to expose an adhesive surface of the frame assembly, positioning the apparatus over a desired external genital region such that the protective layers and frame assembly conform to the user's anatomy and adhere securely to the skin surface, and optionally engaging a detachable coupling mechanism to secure the apparatus relative to a mating frame or support. After use, the method includes removing the apparatus by initiating separation along a tear-away or perforated margin defined along the frame assembly. This implementation allows for consistent, comfortable application and predictable removal of the apparatus while maintaining an effective barrier during use.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a perspective view of a female genitalia protective sheath apparatus of the present invention;

FIG. 2 presents a perspective view showing that the female genitalia protective sheath apparatus of the present invention is flexible and deformable;

FIG. 3 presents another perspective view of the female genitalia protective sheath apparatus of the present invention;

FIG. 4 presents a perspective view showing that a frame cover may be peelable from an adhesive frame according to an embodiment of the female genitalia protective sheath apparatus of the present invention;

FIG. 5 presents a perspective view showing that the female genitalia protective sheath apparatus is going to be used to cover female genitalia according to an embodiment of the present invention;

FIG. 6 presents a perspective view showing that the female genitalia protective sheath apparatus is used to cover the female genitalia according to an embodiment of the present invention;

FIG. 7 presents an exploded view of the female genitalia protective sheath apparatus according to a first embodiment of the present invention;

FIG. 8 presents an exploded view of the female genitalia protective sheath apparatus according to a second embodiment of the present invention;

FIG. 9 presents an exploded view of the female genitalia protective sheath apparatus according to a third embodiment of the present invention;

FIG. 10 presents an exploded view of the female genitalia protective sheath apparatus according to a fourth embodiment of the present invention;

FIG. 11 presents a perspective view showing a ball-pit-type stimulus pad of the female genitalia protective sheath apparatus, wherein little balls within the pad move when pressed to provide a wave-motion stimulus according to an embodiment of the present invention;

FIG. 12 presents a perspective view showing a plurality of string-like protrusions arranged across the stimulus pad surface, the protrusions returning to position after deformation according to an embodiment of the present invention;

FIGS. 13A-13B present a perspective view showing a plurality of column-type protrusions that flex laterally under pressure and return to place upon release according to an embodiment of the present invention;

FIGS. 14A-14B present top and side views illustrating alternative frame geometries of the protective sheath apparatus, including almond-shaped and rounded-end configurations, demonstrating optional variations in frame outline for enhanced fit and retention;

FIGS. 15A-15B present perspective and sectional views illustrating an embodiment incorporating a lower access gap and semi-covered entry region between two protective layers, configured to allow controlled anatomical access while maintaining continuous peripheral coverage;

FIGS. 16A-16B present an enlarged view illustrating an indented or perforated tear-away edge along the perimeter of the sheath apparatus, configured to facilitate user-initiated removal after use without compromising barrier integrity during application; and

FIG. 17 presents a perspective and exploded view illustrating an embodiment including a magnetic-to-metal frame coupling system, wherein a magnetized frame aligns and attaches to a thin metallic cover layer to permit selective detachment and reattachment of the sheath apparatus.

Like reference numerals refer to like parts throughout the several views of the drawings.

### DETAILED DESCRIPTION OF THE PREFERRED EMBODIMENT

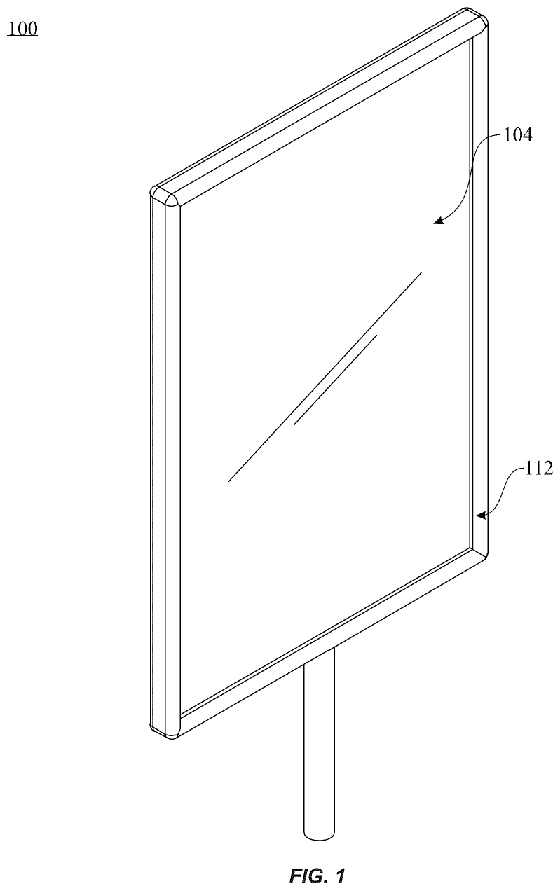

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

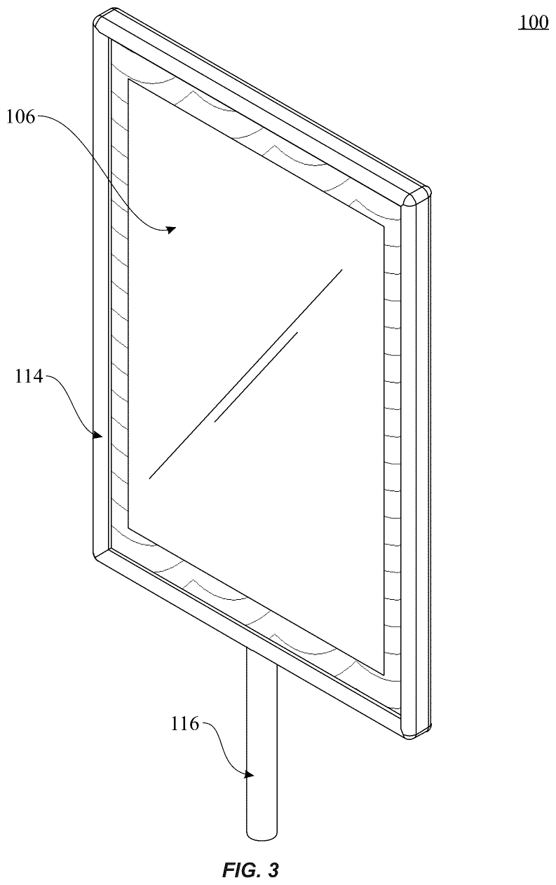

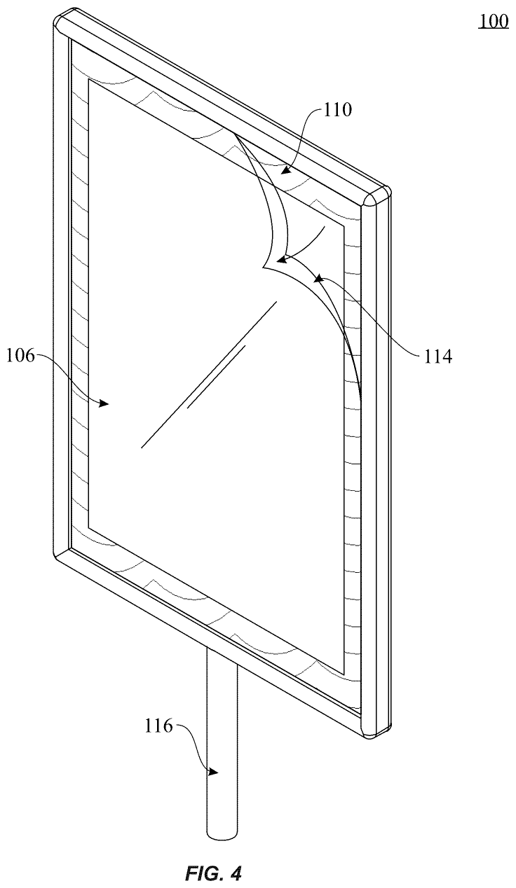

Shown throughout FIGS. 1-17, the present invention is directed toward a female genitalia protective sheath apparatus 100 configured to establish a flexible, anatomically conforming barrier over the external female genitalia. The apparatus 100 is designed to adhere comfortably to a user's skin surface while maintaining a hygienic seal that prevents the exchange of bodily fluids during intimate contact. The structure is lightweight and contoured to follow the natural curvature of the vulvar region, minimizing wrinkling or displacement under movement. In certain configurations, the apparatus may further provide sensory stimulation to enhance user comfort and responsiveness while maintaining complete protection. The system thereby addresses an unmet need for a female-specific prophylactic barrier suitable for oral sexual activity and other close physical interaction.

The apparatus 100 generally comprises a stimulus pad 102, a first protective layer 104, a second protective layer 106, adhesive frames 108 and 110, removable frame covers 112 and 114, and a handle 116. Each component cooperates to create a multilayer assembly that is both elastic and fluid-impermeable. The layers may be arranged symmetrically about a central longitudinal axis such that both sides of the apparatus exhibit equivalent flexibility. The assembly can be folded or rolled for compact packaging without structural damage and unfurls easily when removed from the package. This multilayer configuration enables uniform force distribution and ensures that skin contact remains consistent even during prolonged use.

The stimulus pad 102 forms the internal core of the apparatus 100 and provides both structural support and sensory responsiveness. It may be fabricated from biocompatible polymers including latex, thermoplastic elastomer, polyurethane, silicone, or polyether-based compounds. The material may exhibit a Shore A hardness between 10 and 40 to provide soft tactile compliance while maintaining dimensional stability. The stimulus pad may optionally incorporate plasticizers or soft-touch coatings to enhance surface lubricity and comfort. Additives such as anti-microbial agents or hypoallergenic treatments may also be included to reduce irritation and extend storage life.

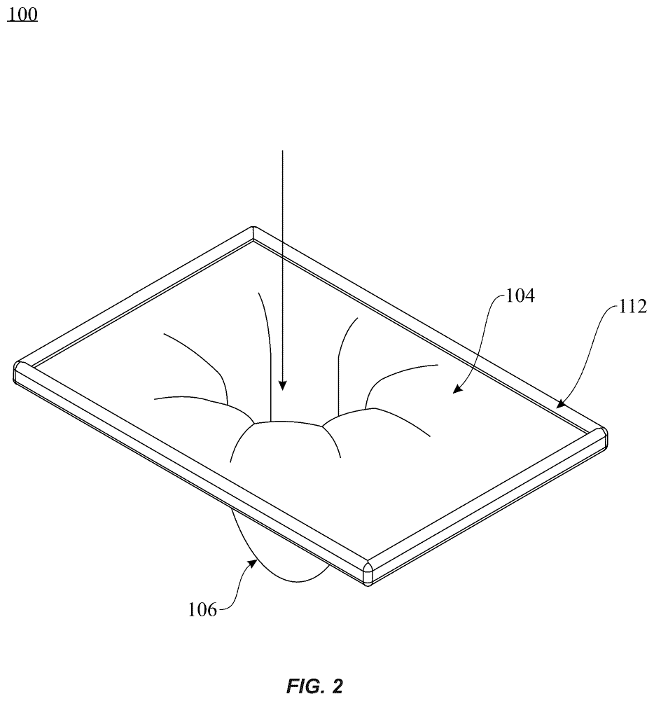

As illustrated in FIG. 2, the stimulus pad 102 is designed to be flexible and deformable such that it conforms to the contours of the vulvar region without tearing or delamination. The pad may incorporate air cavities or micro-bubble inclusions that create localized compressibility and allow the structure to recover immediately after deformation. These cavities may be produced by gas-foaming, solvent expansion, or co-extrusion of a closed-cell film. The resulting elasticity ensures consistent adhesion despite body motion. In certain embodiments, the pad may include reinforcing ribs or peripheral ridges to stabilize its geometry and limit distortion during application.

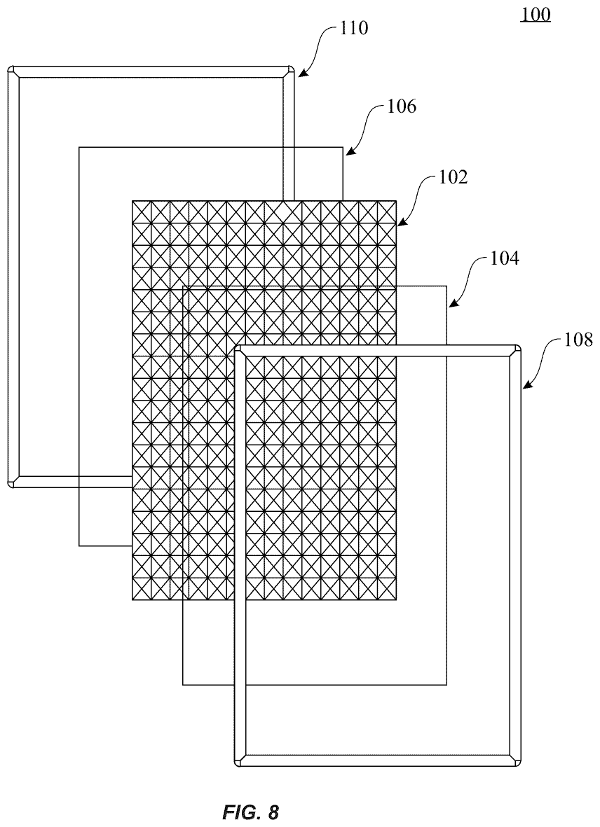

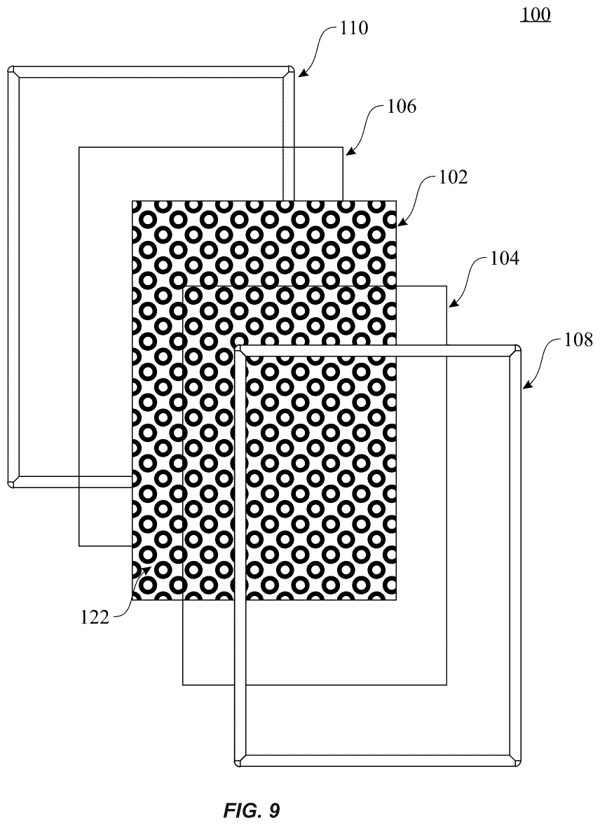

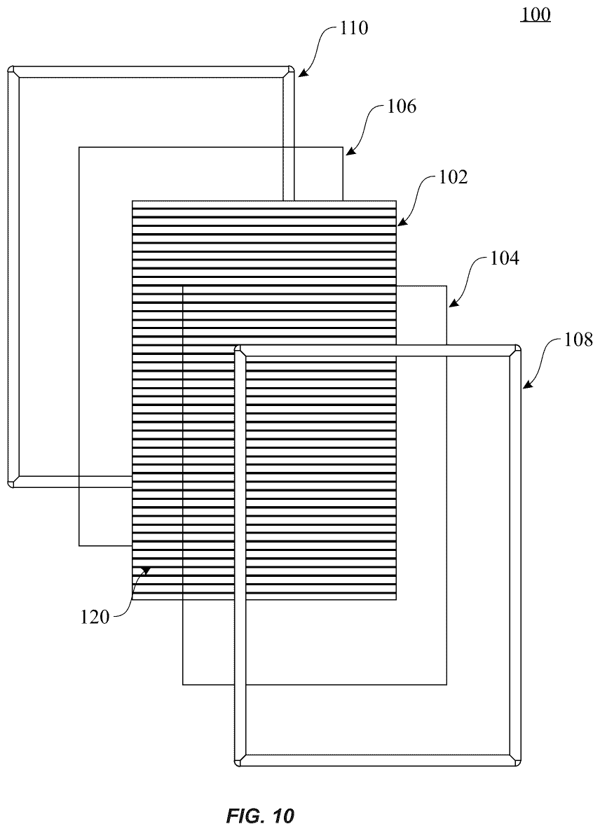

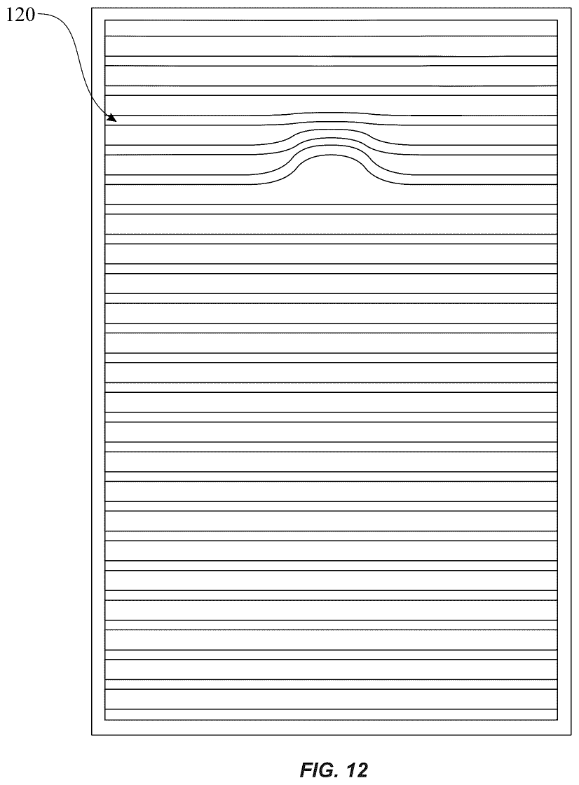

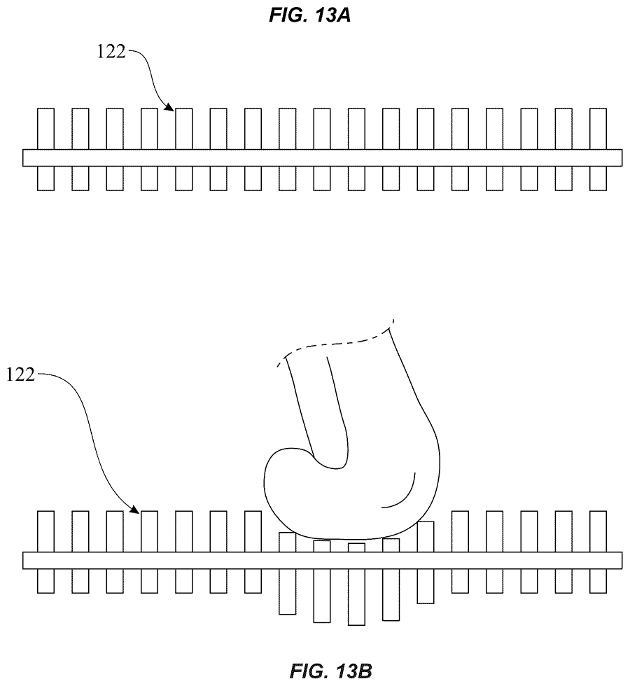

As shown in FIGS. 8-10, the surface of the stimulus pad 102 may include patterned protrusions 120 distributed in controlled arrays such as wave, triangular, string, or dot formations. The height, spacing, and elasticity of these features can be tuned to modify tactile response transmitted through the protective layers. In the configuration of FIGS. 10 and 12, the protrusions 120 are string-like filaments that stretch under pressure and return to their neutral position, generating a micro-vibration effect similar to tongue movement. In the embodiment of FIGS. 9 and 13A and 13B, the protrusions 122 are columnar members that flex laterally under force, providing dynamic feedback when pressure is released. These textures may be molded integrally with the stimulus pad or bonded afterward as a separate textured film. The tactile design offers optional stimulation without compromising the apparatus's barrier integrity.

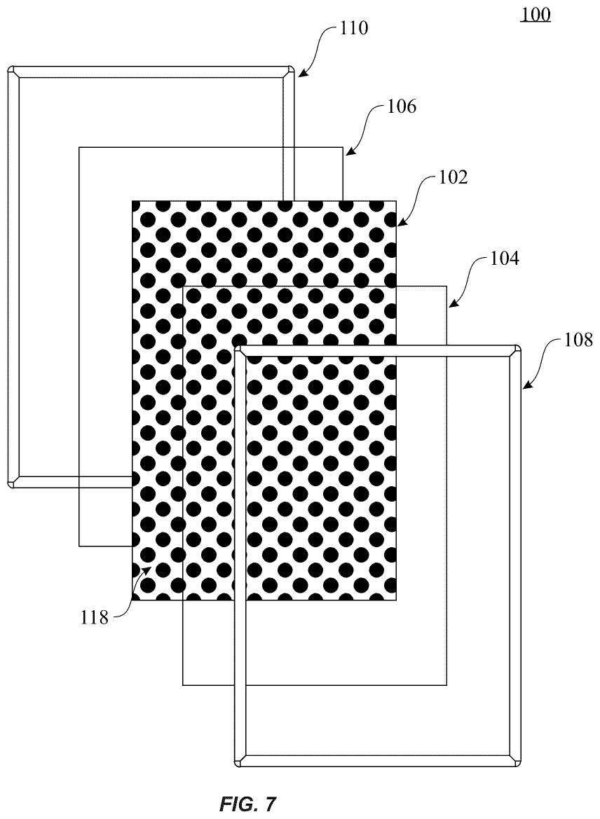

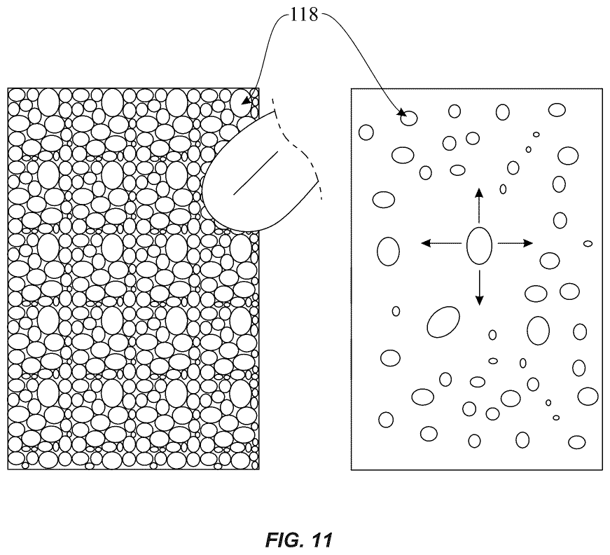

In another configuration shown in FIGS. 7 and 11, the stimulus pad 102 includes a sealed cavity containing a plurality of micro-spheres 118 that roll within the enclosure to generate wave-like kinetic motion. The spheres may be glass, polymeric, or silicone beads coated with a friction-reducing layer. When pressure is applied to the apparatus, the spheres shift collectively, producing dynamic motion and mild vibration across the surface of the pad. The encapsulated volume may include a thin lubricating film that allows free rolling movement while maintaining a complete seal against fluid exchange. This structure enhances comfort and sensitivity during use while retaining all hygienic properties.

The first protective layer 104 is superposed on one side of the stimulus pad 102 and may be composed of a transparent or translucent polymer film. Suitable materials include polyurethane, polyethylene, polyester, or laminated co-polymers optimized for medical adhesion. The layer thickness may range from 20 to 100 micrometers to balance tear strength with flexibility. The layer may be thermally bonded or ultrasonically welded to the stimulus pad, producing a fluid-tight seal at the perimeter. The film may further include anti-fog or moisture-management coatings that preserve optical clarity throughout use.

Similarly, the second protective layer 106 is disposed opposite the first protective layer 104 to enclose the stimulus pad 102. The second layer may be formed of identical or complementary polymeric materials providing equivalent flexibility and transparency. Both protective layers may be pre-stretched during lamination to create a tensioned assembly that conforms smoothly to the user's skin. When assembled, the multilayer arrangement forms a sealed, homogeneous barrier structure that prevents penetration by liquids or microorganisms. The interface between layers may optionally contain a lubricious or textured intermediate coating to facilitate dynamic motion between the internal surfaces.

Along the outer boundary of the apparatus 100, adhesive frames 108 and 110 are provided to secure the protective layers 104 and 106 to the user's skin. The adhesives may be medical-grade acrylic, silicone, or hydrocolloid formulations chosen for biocompatibility and moisture resistance. Each frame may have a width between 5 and 15 millimeters to create a sufficient sealing footprint around the perimeter. The adhesive surface may incorporate micro-perforations that release trapped air during application, preventing wrinkles and ensuring smooth contact. In some embodiments, the frames may include color indicators that change hue upon successful adhesion, confirming proper placement.

To maintain sterility and prevent premature adhesion, frame covers 112 and 114 are removably attached to the corresponding adhesive frames 108 and 110. These covers may be constructed from paper, polymeric film, or foil laminate with a low-tack silicone release coating. Each cover may include pull-tabs or textured corners to assist removal without contaminating the adhesive surface. During manufacturing, the apparatus 100 may be assembled and sterilized within a controlled environment, after which the covers are applied and sealed in a hermetic package. The covers protect the adhesive until the apparatus is ready for use and also contribute to maintaining the overall flat geometry of the device during storage.

A handle 116 extends laterally from the perimeter of the apparatus 100, allowing controlled manipulation during placement and removal. The handle may be integrally molded with the protective layers or bonded after assembly using an adhesive or heat-seal process. It may have a ribbed or dimpled texture that enhances grip, particularly in moist conditions. The handle 116 ensures that the user can align and adjust the apparatus without contacting the central barrier surface, preserving sterility. It also provides leverage during removal, reducing stress on the adhesive interfaces.

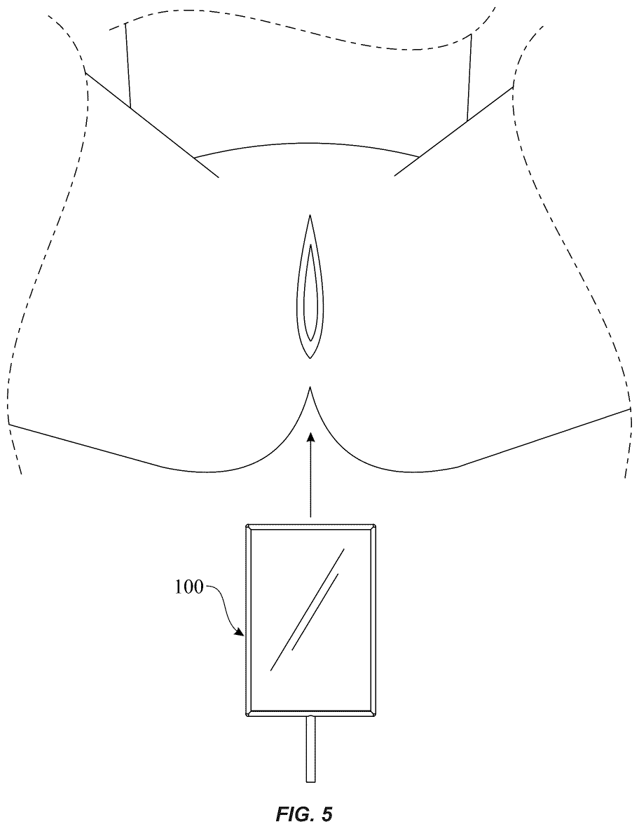

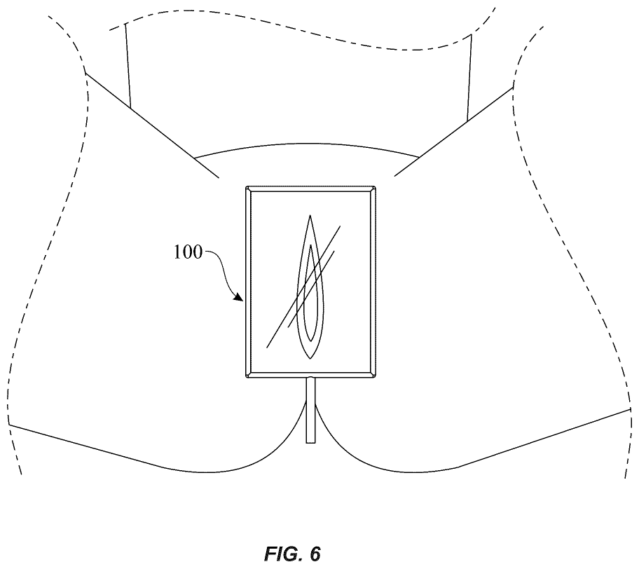

During use, as illustrated in FIGS. 5 and 6, the apparatus 100 is positioned so that one of the protective layers, typically the first protective layer 104, contacts the user's skin surrounding the external genitalia. The opposite surface faces outward toward the partner, forming a protective barrier. Once the frame covers 112 and 114 are peeled away, the adhesive frames 108 and 110 are pressed gently onto the skin to achieve a complete seal. The elasticity of the materials allows the apparatus to conform dynamically to movement while maintaining adhesion. This configuration prevents direct exchange of fluids such as saliva and vaginal secretions, significantly reducing the risk of infection while preserving comfort.

Removal is accomplished by grasping the handle 116 and peeling the apparatus gently away from the skin, allowing the adhesive to release uniformly. The apparatus may then be folded and disposed of in accordance with sanitary waste protocols. The materials are designed to detach without residue, leaving the skin clean and free of irritation. This disposability makes the apparatus 100 suitable for single-use applications in both personal and clinical settings.

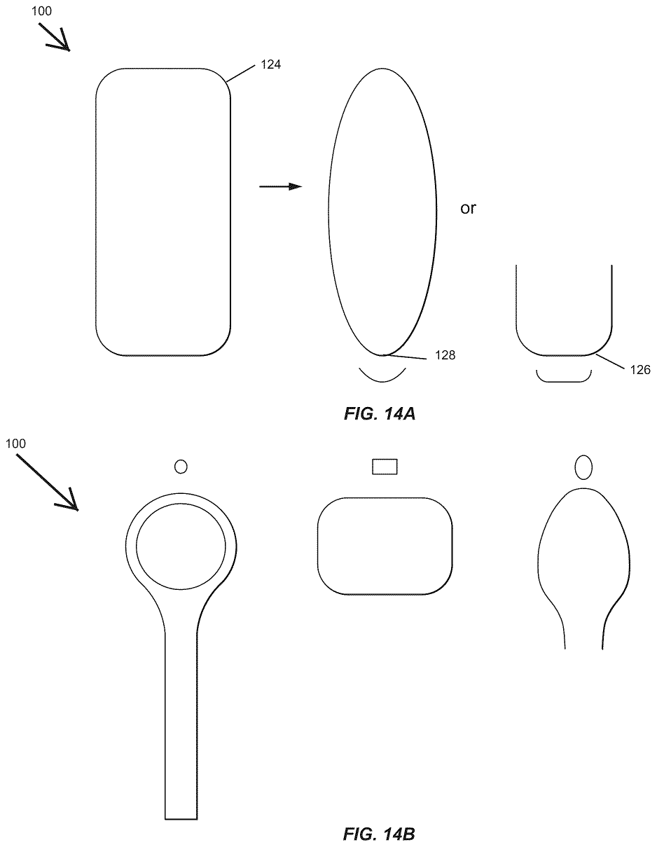

Additional embodiments introduce new structural features to improve conformity, functionality, and user interaction. As shown in FIGS. 14A and 14B, the apparatus 100 may incorporate a geometrically contoured frame 124 defining an almond-shaped outline. The almond geometry mirrors the natural profile of the vulvar area, enhancing sealing efficiency and positional stability. The lower end of the frame 124 may terminate in a flatted edge 126 that distributes pressure evenly across the perineal region. The frame 124 may be die-cut from a flexible polymer sheet or pre-molded using a thermoforming process to achieve consistent curvature and edge smoothness.

The upper region of the frame 124 may include a rounded end 128 configured to accommodate varying anatomical contours. The frame and adhesive layer may be co-extruded or laminated to create a continuous bonded interface that resists separation during use. This contoured design minimizes shear forces and reduces the likelihood of peeling. In addition to improved fit, the frame 124 provides aesthetic uniformity and may include subtle surface embossing that indicates correct orientation to the user. The geometry also serves as a structural reinforcement, preventing deformation when the apparatus is applied or removed.

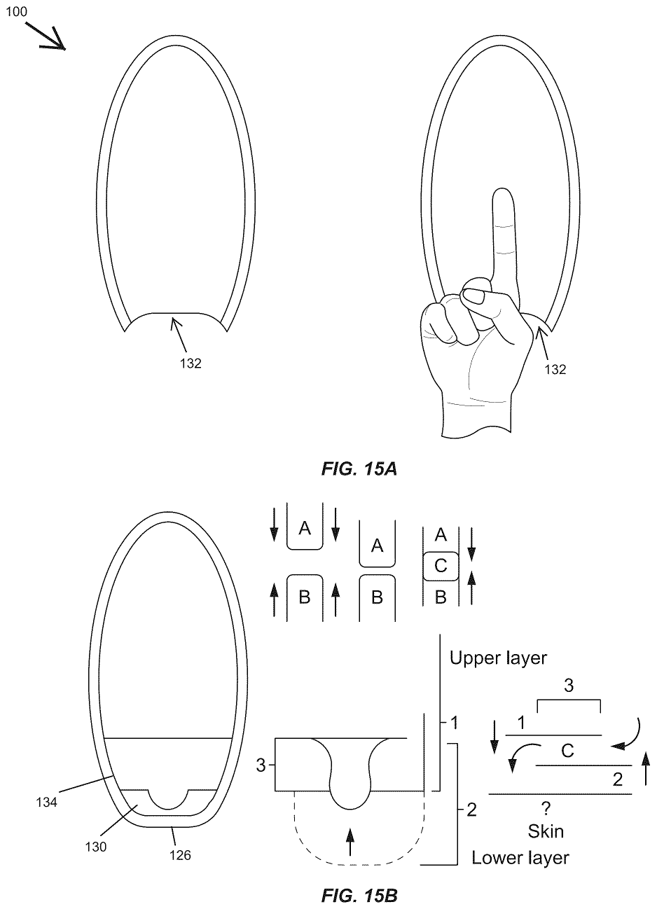

As illustrated in FIGS. 15A and 15B, the apparatus 100 may include a lower access region 130 positioned near the flatted edge 126. The access region 130 creates a small, controlled gap or opening between adjacent portions of the protective layers 104 and 106. This opening allows partial anatomical access for specific uses such as therapeutic procedures or selective stimulation without removing the entire apparatus. The width of the access region can vary based on application but remains small enough to preserve continuous coverage of surrounding skin. A semi-covered entrance 132 formed by overlapping edges of the protective layers may optionally be provided to maintain a sealed yet flexible flap.

A reinforcement border 134 may surround the access region 130 to prevent tearing or delamination. The reinforcement may consist of a thicker polymer segment or an embedded mesh filament that strengthens the local area while maintaining flexibility. It may also function as a tactile guide that allows the user to locate the access region by touch without visual confirmation. The reinforcement border can be integrated during molding or added as a secondary insert bonded with thermal or ultrasonic energy. Such reinforcement ensures the structural durability of the apparatus even under repeated flexion or manipulation.

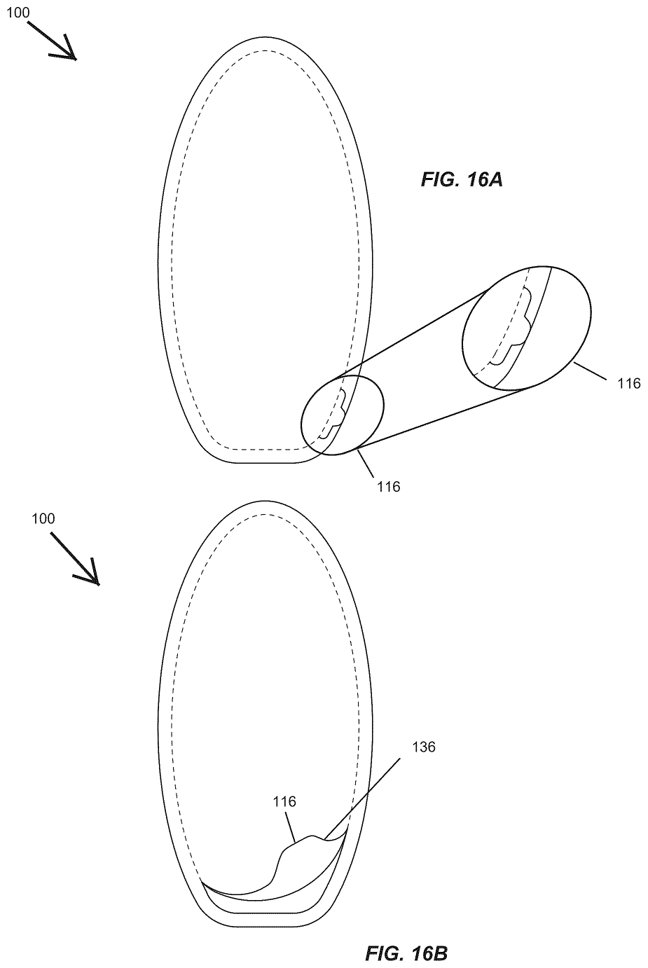

FIGS. 16A-16B show another embodiment wherein the apparatus 100 incorporates a tear-away perimeter 136 disposed along one or more sides of the frame 124. The tear-away perimeter 136 may include micro-perforations, indentations, or laser-scored lines that permit predictable separation when a controlled peeling force is applied. The user can grasp the handle 116 or a designated pull-tab adjacent to the perforated edge to initiate removal. The perforations are engineered to resist tearing during use yet yield easily when intentionally pulled, ensuring safe and effortless removal. This feature improves user convenience and eliminates the need for high adhesive strength that could cause discomfort upon detachment.

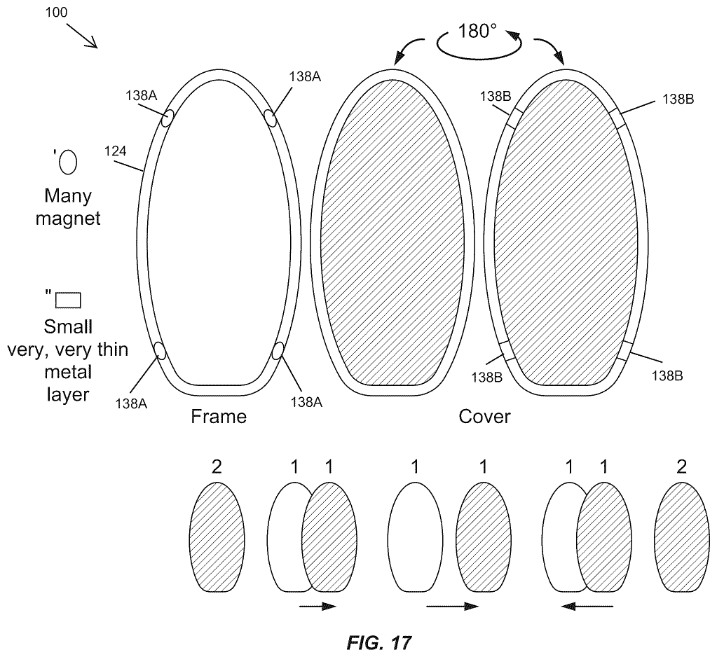

As depicted in FIG. 17, another embodiment features a detachable coupling system 138 allowing the apparatus 100 to be secured magnetically or mechanically to an external frame. The coupling system may include a magnetic element 138a embedded within the frame 124 and a metallic counterpart 138b disposed on a mating frame or garment. The magnetic components may be neodymium or ferrite magnets sealed within a polymer casing to ensure corrosion resistance. Polarized alignment ensures automatic orientation, enabling quick and repeatable positioning. This detachable system allows the apparatus to be anchored without relying solely on adhesives and can facilitate use in conjunction with undergarments or medical supports.

A method of applying the female genitalia protective sheath apparatus 100 may include providing a sterilized apparatus within sealed packaging, removing the frame covers 112 and 114 to expose the adhesive surfaces, and positioning the apparatus such that the contoured frame 124 aligns with the external genitalia. The handle 116 is grasped to guide placement, and light pressure is applied along the adhesive periphery to secure adhesion. During use, the apparatus remains stable and conforms to body movement, while the stimulus pad 102 transmits mild tactile sensations through the protective layers.

The method may further include optionally engaging the coupling system 138 by bringing the apparatus 100 into proximity with a corresponding magnetic or mechanical frame. Once secured, the user may engage in activity while the apparatus maintains a complete seal around the vulvar region. Upon completion, the user grasps the handle 116 and pulls along the tear-away perimeter 136 to remove the apparatus smoothly. The structure releases evenly without discomfort, and the used unit may be folded and discarded in accordance with sanitary disposal practices.

In alternative applications, the same method allows partial opening of the semi-covered entrance 132 to introduce diagnostic instruments or therapeutic devices in a clinical environment. The barrier remains intact around the surrounding tissue, maintaining sterility and preventing cross-contamination. The apparatus may thus serve both recreational and medical purposes, adapting to diverse usage scenarios with minimal modification.

While specific embodiments have been described in connection with FIGS. 1-17, numerous variations fall within the scope of the invention. The protective layers 104 and 106 may incorporate additional surface coatings such as lubricants, pH-balancing agents, or conductive films for heating or sensing applications. The adhesive frames 108 and 110 may be reformulated using hydrogel or pressure-sensitive adhesives suitable for water immersion. The magnetic coupling system 138 may be replaced with micro-clips, suction elements, or reversible electrostatic adhesion. The shape of the frame 124 may vary from almond-shaped to elliptical or polygonal depending on anatomical requirements. Any of the structural features described herein—such as the access region 130, reinforcement border 134, tear-away perimeter 136, and coupling system 138—may be implemented individually or in combination, providing modularity and adaptability for both consumer and medical implementations. The invention therefore encompasses all such modifications, equivalents, and design alternatives consistent with the appended claims.

Since many modifications, variations, and changes in detail can be made to the described preferred embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

What is claimed is:

1. A female genitalia protective sheath apparatus comprising:
a stimulus pad configured to provide a tactile interface;
a first protective layer and a second protective layer each superposed onto opposing sides of the stimulus pad;
a frame assembly coupled to a periphery of at least one of the first and second protective layers;
a removable cover attached to the frame assembly to preserve sterility prior to use; and
a handle laterally connected with the stimulus pad and at least one of the first or second protective layers;
wherein the frame assembly defines an enhanced peripheral architecture comprising:
a geometrically contoured frame having at least one almond-shaped, rounded-end, or flatted-end configuration dimensioned to improve anatomical conformity and positional retention when applied to a user's skin surface;
an access region formed between adjacent portions of the first and second protective layers proximate a lower end of the frame assembly, the access region providing a controlled passageway that enables limited anatomical access while maintaining continuous peripheral barrier coverage;
a tear-away perimeter defined by an indented or perforated margin along at least a portion of the frame assembly, the tear-away perimeter being configured to facilitate predictable user-initiated removal of the apparatus following use;
a detachable coupling mechanism integrated within the frame assembly, the coupling mechanism comprising magnetic and metallic components configured to enable selective attachment and reattachment of the protective sheath apparatus to a mating frame or structural support; and
wherein the first protective layer, the second protective layer, and the stimulus pad are each formed from flexible, biocompatible materials that conform to external female anatomy and collectively define a barrier apparatus configured for hygienic protection during sexual activity.

2. The female genitalia protective sheath apparatus of claim 1, wherein the geometrically contoured frame defines a generally almond-shaped outline dimensioned to follow an external contour of a user's vulvar region to improve retention and coverage.

3. The female genitalia protective sheath apparatus of claim 1, wherein the geometrically contoured frame defines a rounded-end configuration having a flatted lower edge adapted to provide enhanced comfort when positioned between adjacent body surfaces.

4. The female genitalia protective sheath apparatus of claim 1, wherein the frame assembly is formed of a semi-rigid polymeric material that provides elastic memory sufficient to maintain shape after deformation during application.

5. The female genitalia protective sheath apparatus of claim 1, wherein the access region is disposed at a lower portion of the frame assembly and defines an open gap extending between the first and second protective layers to enable controlled anatomical access.

6. The female genitalia protective sheath apparatus of claim 1, wherein the access region comprises a semi-covered entrance defined by overlapping edge portions of the first and second protective layers to permit limited tactile contact while maintaining peripheral sealing.

7. The female genitalia protective sheath apparatus of claim 1, wherein the access region further includes an interior reinforcement border configured to prevent tearing or delamination of the protective layers during use.

8. The female genitalia protective sheath apparatus of claim 1, wherein the tear-away perimeter comprises a series of perforations extending along a defined segment of the frame assembly to guide controlled removal of the apparatus after use.

9. The female genitalia protective sheath apparatus of claim 1, wherein the tear-away perimeter comprises an indented score line formed within the frame assembly to initiate predictable rupture under manual peeling force.

10. The female genitalia protective sheath apparatus of claim 1, wherein the detachable coupling mechanism comprises a magnetic element disposed within the frame assembly and a complementary metallic element disposed on a mating frame or support structure.

11. The female genitalia protective sheath apparatus of claim 10, wherein the magnetic element is selected from neodymium, samarium-cobalt, or ferrite materials encapsulated within a polymeric housing.

12. The female genitalia protective sheath apparatus of claim 10, wherein the magnetic element is arranged to provide polarized alignment that automatically seats the protective sheath apparatus into a defined operational position relative to the mating frame.

13. The female genitalia protective sheath apparatus of claim 1, wherein the frame assembly further comprises an adhesive layer disposed beneath the magnetic or metallic components to enhance sealing contact with the user's skin surface.

14. The female genitalia protective sheath apparatus of claim 1, wherein the frame assembly, protective layers, and stimulus pad are bonded together by a lamination or heat-sealing process to provide a unified barrier structure.

15. The female genitalia protective sheath apparatus of claim 1, wherein the first and second protective layers are formed from transparent or translucent biocompatible polymeric films to maintain visual monitoring of the covered area.

16. The female genitalia protective sheath apparatus of claim 1, wherein the stimulus pad includes a textured inner surface configured to provide tactile stimulation through the protective layers during contact.

17. The female genitalia protective sheath apparatus of claim 1, wherein the handle is integrally formed with the frame assembly as a single continuous molded component to facilitate grasping and removal of the apparatus.

18. The female genitalia protective sheath apparatus of claim 1, wherein the frame assembly, protective layers, and removable cover collectively define a disposable barrier unit sterilized and packaged for single-use application.

19. A female genitalia sheath comprising:
a stimulus pad configured to provide a tactile interface;
a first protective layer and a second protective layer each superposed onto opposing sides of the stimulus pad;
a frame assembly coupled to a periphery of at least one of the first and second protective layers;
a removable cover attached to the frame assembly to preserve sterility prior to use; and
a handle laterally connected with the stimulus pad and at least one of the first or second protective layers;
wherein the frame assembly defines a geometrically contoured peripheral architecture comprising an almond-shaped outline having a flatted lower end dimensioned to conform to external female anatomy and improve positional stability;
wherein a lower portion of the frame assembly defines a semi-covered access region formed by overlapping edge portions of the first and second protective layers to permit limited anatomical access while maintaining continuous peripheral coverage;
wherein at least a segment of the frame assembly includes a perforated tear-away margin configured to allow predictable user-initiated removal following use; and
wherein the frame assembly further comprises a detachable magnetic coupling mechanism including a magnet embedded within the frame assembly and a complementary metallic element disposed on a mating frame or support to enable selective attachment and reattachment of the apparatus.

20. A method of applying a protective sheath apparatus to provide a hygienic barrier
over external female genitalia, the method comprising:
providing a protective sheath apparatus comprising a stimulus pad positioned between a first protective layer and a second protective layer, the protective layers being coupled to a peripheral frame assembly;
removing a removable cover from the frame assembly to expose an adhesive surface;
positioning the frame assembly and the protective layers over an external genital region such that the apparatus conforms to the user's anatomy and adheres to a skin surface;
engaging a detachable coupling system to secure the apparatus relative to a mating frame or support; and
after use, removing the apparatus by initiating separation along a user-separable periphery of the frame assembly.
